(19) United States
(12) Patent Application Publication
(10) Pub. No.: US 2025/0352036 A1
(43) Pub. Date: Nov. 20, 2025

(54) METHOD FOR TRANSPORTING AND VERIFYING CALIBRATION VALUES ASSOCIATED WITH SENSORS OF AN ENDOSCOPE, AND DEVICE FOR HOUSING AND TRANSPORTING THE ENDOSCOPE

(71) Applicant: ATLAS ENDOSCOPY LIMITED, LEEDS (GB)

(72) Inventors: Bruno SCAGLIONI, LEEDS (Yorkshire) (GB); Donato MAZZEO, LEEDS (Yorkshire) (GB); Pietro VALDASTRI, LEEDS (Yorkshire) (GB)

(21) Appl. No.: 19/128,540

(22) PCT Filed: Nov. 7, 2023
(86) PCT No.: PCT/IB2023/061215

§ 371 Date: May 8, 2025

(30) Foreign Application Priority Data
Nov. 10, 2022 (IT) 102022000023211

(51) Int. Cl. A61B 1/00; A61B 1/04; A61B 5/06; A61B 34/20; A61B 34/30; A61M 25/00

(52) U.S. Cl. CPC A61B 1/00057; A61B 1/04; A61B 5/062; A61B 34/30; A61B 2034/2048; A61B 2034/301; A61M 25/002

(57) ABSTRACT

The invention relates to a method for transporting and verifying calibration values associated with sensors accommodated in an endoscope usable in a magnetically guided endoscopic system. The method includes providing an endoscopic robotic system comprising:
an endoscope including one or more sensors;
a magnetic field source for providing a reference magnetic field value to the endoscope;
an electronic processing unit;
a reading unit connected to the endoscope to acquire data representative of measurements made by said one or more sensors to be sent to the electronic processing unit;
a reading unit of calibration values associated with said one or more sensors, connected to the endoscope to acquire said calibration values to be sent to the electronic processing unit.

The method further comprises the steps of:
providing an endoscope housing and transport device having a body adapted to house the endoscope;
coupling and making integral the endoscope with the body of the housing and transport device, preventing a mutual movement thereof;
placing the housing and transport device in a predetermined position of the endoscopic robotic system to provide the endoscope with the reference magnetic field value generated by the magnetic field source;
acquiring, by the electronic processing unit:
   the calibration values associated with said one or more sensors, first data representative of measurements made by said one or more sensors when the reference magnetic field value is applied to the endoscope in the predetermined position of the endoscopic robotic system;
   reference data representative of correct measurements made by the one or more sensors when the reference magnetic field value is applied to the endoscope in the predetermined position of the endoscopic robotic system;
applying the calibration values to the first acquired data to generate first calibrated data;
comparing the first calibrated data with the reference data;
returning, by said electronic processing unit, based on such a comparison:
   first information, indicating that the calibration values associated with the one or more sensors are correct, or
   second information indicating that the calibration values associated with the one or more sensors are incorrect.

### TECHNOLOGICAL BACKGROUND OF THE INVENTION

### Field of Application

The present invention generally relates to magnetically guided endoscopic systems usable in the medical field. In particular, the invention relates to a method for transporting and verifying calibration values associated with sensors accommodated in an endoscope, before using such an endoscope in a magnetically guided endoscopic system.

The present invention also relates to a device for housing and transporting the endoscope usable to perform the aforesaid method.

### Prior Art

As known, a magnetically guided endoscopic robotic system consists of a robotic platform supporting in the end part thereof one or more electronically controllable magnetic field sources, in terms of generated field and/or position and orientation of the field itself, and of a capsular endoscopic element, for example connected by means of an electrical connection (wired) to the robotic platform, containing a magnetic field source therein.

Such a capsular endoscopic element or endoscope is insertable into a patient's natural cavity, for example in the gastrointestinal tract, through the natural sphincters to perform a diagnostic assessment on the patient. In particular, under the action of the magnetic field generated by the one or more controllable magnetic field sources of the robotic platform, it is possible to orient, locate and control the movement of the endoscope within the patient's gastrointestinal tract.

U.S. Pat. No. 11,122,965 B2 and other similar known technical solutions describe an endoscopic capsule of known type usable in a magnetically guided endoscopic system. Such an endoscopic capsule comprises a permanent magnet inside the capsule and a plurality of sensors, including:
one or more magnetic field sensors (MFS), for example one or more Hall-effect sensors;
an Inertial Measurement Unit (IMU) which generally comprises an accelerometer and/or a gyroscope.

An endoscopic capsule can further comprise a camera, usable to allow an operator to view sections of the patient's gastrointestinal tract and to manually control the movement of the capsule, in addition to enabling any automatic image processing, such as automatic learning and navigation algorithms.

As known, in order to ensure the correctness of the measurements made, the aforesaid sensors equipping the endoscope require a calibration before using the endoscope itself.

In addition, if the aforesaid calibration is carried out in a place, for example in the plant where the endoscope was manufactured, other than the place where the endoscope itself will then be used, for example the room accommodating the robotic platform with which the diagnostic investigation is carried out on the patient, or if such calibration was carried out a long time before use, it is necessary to provide for some further contrivances to ensure the correct operation of the endoscope.

In particular, after the calibration performed in the manufacturing plant, it is required that the calibration values calculated with the calibration operation can be appropriately recorded and transported together with the endoscope.

Moreover, before performing the diagnostic investigation on the patient, it is required that such calculated calibration values can be examined to verify that they are still valid. This allows discriminating whether the endoscope can be used for diagnostic investigation by the magnetically guided robotic system or should be discarded, or whether calibration values should be recalculated.

Nowadays, there are no solutions specifically designed which allow simultaneously meeting the aforesaid needs, so as to ensure both the recording and transport of the calculated calibration values and the verification of the validity of such calibration values.

### SUMMARY OF THE INVENTION

Therefore, it is the object of the present invention to provide a method for transporting and verifying calibration values associated with sensors accommodated in an endoscope, before using such an endoscope in a magnetically guided endoscopic system, which ensures both the recording and transport of the calculated calibration values and the verification of the validity of such calibration values in a reliable and simple manner.

Such an object is achieved by a method for transporting and verifying calibration values associated with sensors accommodated in an endoscope according to claim 1.

The aforesaid verification of the validity of the calibration values of the endoscope sensors allows, in particular, discriminating if the endoscope is usable, or if it needs recalibration or must be disposed of as it is not usable.

Preferred and advantageous embodiments of the method for transporting and verifying calibration values associated with sensors of an endoscope are the subject of the dependent claims.

The present invention also relates to a device for housing and transporting the endoscope according to claim 19, usable to perform the aforesaid method.

In particular, such a housing and transport device is configured to protect the endoscope during transport and to keep the endoscope clean, preventing direct contact with dirty surfaces in the aforesaid verification step and before use.

### BRIEF DESCRIPTION OF THE DRAWINGS

Further features and advantages of the invention will be apparent from the following description of preferred embodiments thereof, given by way of non-limiting indication, with reference to the accompanying drawings, in which:

FIG. 1 diagrammatically shows an embodiment of a magnetically guided endoscopic robotic system usable to perform the method for transporting and verifying calibration values associated with sensors accommodated in an endoscope of the present invention;

FIGS. 2A, 2B, 2C show a front perspective view in a closed configuration, a rear perspective view in a closed configuration, and a front perspective view in an open configuration, respectively, of an example of a device for housing and transporting the endoscope usable in the system in FIG. 1;

FIGS. 3A, 3B, 3C show an exploded front perspective view, an exploded rear perspective view, and an exploded sectional side view, respectively, of an assembly comprising the device for housing and transporting the endoscope in FIG. 3C and an interface element housed in a robotic platform to allow coupling the housing and transport device to the robotic platform;

FIG. 4 diagrammatically shows a perspective view of a robotic platform usable in the system in FIG. 1;

FIGS. 5 and 6 show, in front perspective view and in section, the assembly comprising the interface element housed in the robotic platform and the device for housing and transporting the endoscope in FIG. 3A in an assembled configuration;

FIG. 7 diagrammatically shows the elements accommodated inside an endoscope placed inside the device for housing and transporting the endoscope of the invention;

FIG. 8 shows, with a flow diagram, the operating steps of the method for transporting and verifying the calibration values associated with sensors accommodated in an endoscope of the present invention;

FIGS. 9A, 9B, 9C, 9D show, with flow diagrams, embodiments of some operating steps of the transport and verification method in FIG. 8.

Similar or equivalent elements in the aforesaid figures are indicated by the same reference numerals.

### DETAILED DESCRIPTION

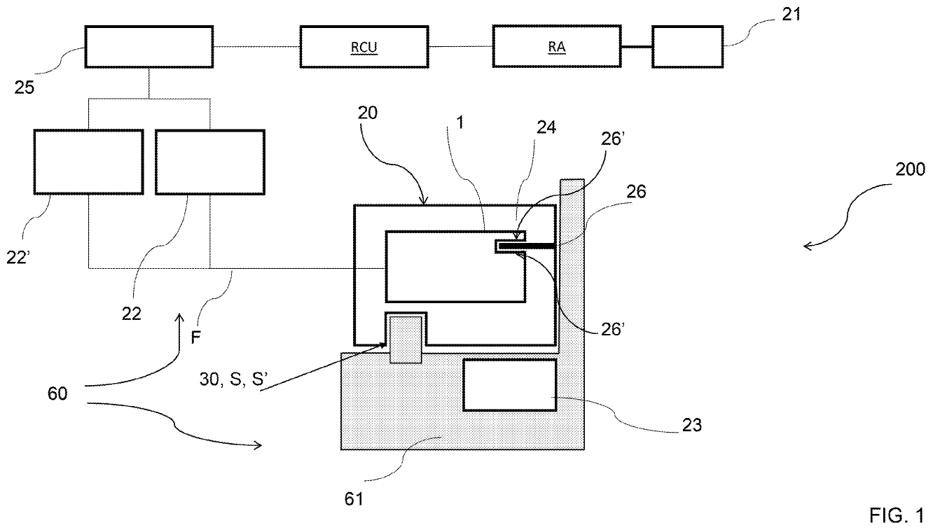

With reference to FIG. 1, a magnetically guided endoscopic system usable to perform the method 100 for transporting and verifying the calibration values associated with sensors accommodated in an endoscope 1 of the present invention is indicated overall with reference numeral 200.

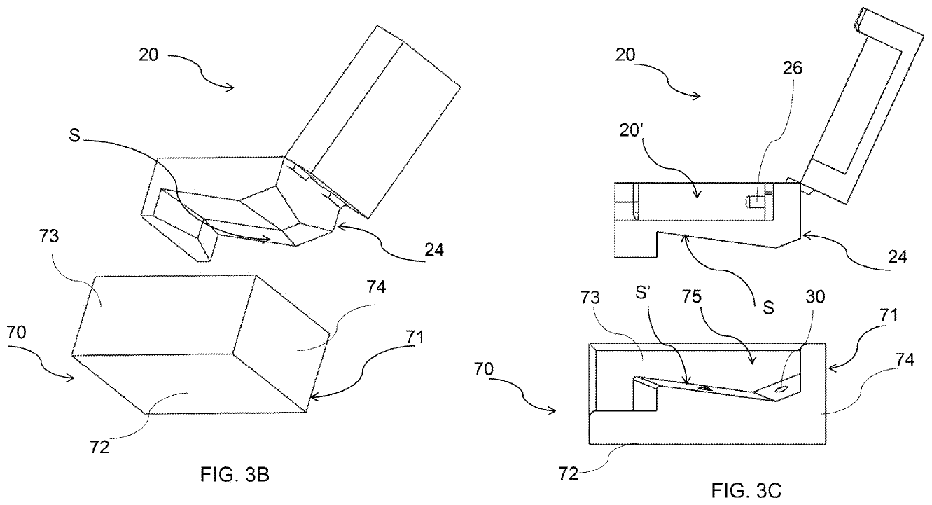

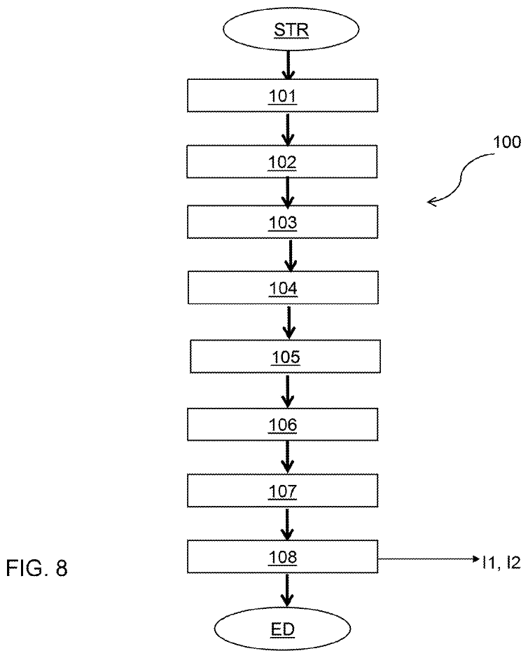

The aforesaid magnetically guided endoscopic system 200 consists, as known, of a robotic platform, for example the robotic platform 60 diagrammatically shown in FIG. 4, which supports in the end part thereof one or more magnetic field sources 21, the position and/or intensity of which is electronically controllable and of a capsular endoscopic element or endoscope 1. Such an endoscope 1 is, for example, connected by at least one electric cable F to the robotic platform 60 and comprises a permanent magnetic field source therein, in particular a permanent magnet 5, shown in FIG. 7.

The aforesaid endoscope 1 is insertable, for example, into the gastrointestinal tract of a patient to perform a diagnostic assessment on the patient.

Such a magnetically guided endoscopic system 200 or, more simply, system comprises the above-mentioned endoscope 1 including one or more sensors 2, 3, 4, 6 requiring calibration.

In the embodiment in FIG. 7, such one or more sensors of the endoscope 1 comprise:
an accelerometer 2,
a gyroscope 3,
one or more magnetic field sensors 4 (MFS).
   Such one or more magnetic field sensors 4 are operatively associated with the permanent magnet 5 housed inside the endoscope 1. Such magnetic field sensors 4 are, for example, one or more Hall-effect sensors.

In a further embodiment, the aforesaid one or more sensors of the endoscope 1 further comprise a camera 6.

The system 200 further comprises an electronic processing unit 25, for example a microprocessor (Central Processing Unit or CPU) and a magnetic field source 21, 23 for providing a reference magnetic field value to the endoscope 1.

In a first embodiment, such a magnetic field source is embodied in a magnetic field source 21 controlled by the electronic processing unit 25 to provide a reference magnetic field value to the endoscope 1.

In particular, the electronic processing unit 25 is configured to control a respective control unit RCU of a robotic arm. Such a robotic arm control unit RCU is configured to move a robotic arm RA supporting the aforesaid controlled magnetic field source 21 outside the endoscope 1.

In a different embodiment, such a magnetic field source is a permanent magnet 23 housed in a base portion 61 of the robotic platform 60 of the endoscopic robotic system 200.

The system 200 further comprises a reading unit 22 connected to the endoscope 1 and configured to acquire data representative of measurements made by the aforesaid one or more sensors 2, 3, 4, 6 of the endoscope to be sent to the electronic processing unit 25.

In the embodiment in FIG. 1, such a reading unit 22 is an electronic unit outside the endoscope 1 and operates to acquire the measurement data from the sensors 2, 3, 4, 6 in analog form and to convert them into corresponding digital data to be transferred to the electronic processing unit 25 configured to process them.

In a different embodiment (not shown in the figures), such a reading unit 22 is a unit equipping the endoscope 1 itself.

In addition to the above-mentioned structural components, the system 200 also comprises a calibration value reading unit 22′ associated with said one or more sensors 2, 3, 4, 6 of the endoscope 1, connected to the endoscope 1 to acquire such calibration values to be sent to the electronic processing unit 25.

The system 200 further comprises an endoscope housing and transport device 20 having a body 24 adapted to house the endoscope 1 and reversible coupling means 26, 26′ for reversibly coupling the endoscope 1 to the body 24 of the housing and transport device 20.

In particular, such reversible coupling means 26, 26′ are configured to couple and make integral the endoscope 1 with the body of the housing and transport device 20 preventing a mutual movement thereof.

The endoscopic robotic system 200 further comprises means 30, S, S′ for removably coupling the housing and transport device 20 to the robotic platform 60 of the endoscopic robotic system 200 in a predetermined position.

Such removable coupling means 30, S, S′ can be mechanical, magnetic, or a combination of both.

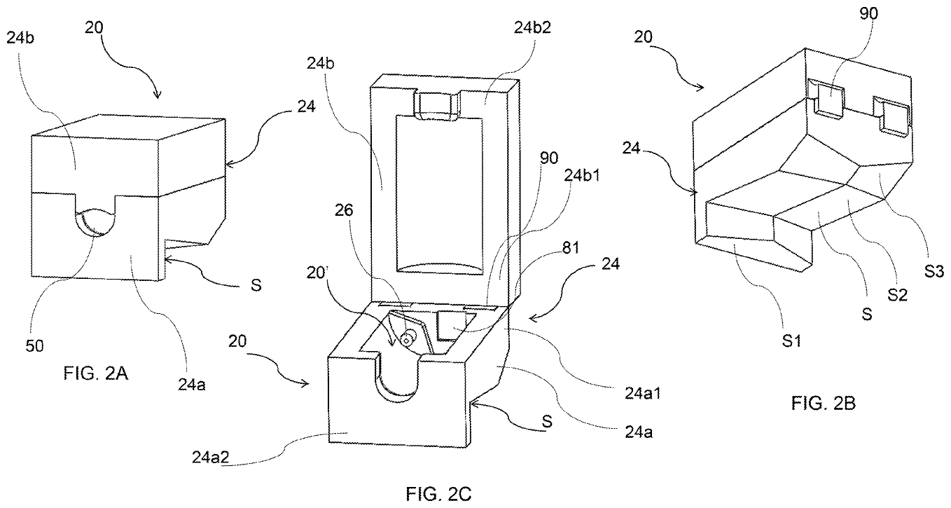

With reference to the embodiment in FIGS. 2A-2C, the body 24 of the housing and transport device 20 of the invention comprises a coupling surface S configured to engage, by a mechanical shape coupling, the magnetically guided endoscopic robotic system 200.

In greater detail, the body 24 of the housing and transport device 20 has a box-like shape to delimit an internal cavity 20′ for housing the endoscope 1. Such a body 24 comprises a first 24a and a second 24b body portion or first and second half-shell. The first body portion 24a has a first end 24a1 connected, for example, by a hinge 90, in particular two hinges, to a respective first end 24b1 of the second body portion 24b. As an alternative to hinges, it is possible to use two welded/melted/glued elements which once opened indicate that the device has been used and thus cleaning is no longer ensured.

Each of such first 24a and second 24b body portions has a respective second free end 24a2, 24b2 opposite to the aforesaid first end.

The aforesaid hinge 90 allows a mutual movement of the first 24a and second body portions 24b between a closed position of the housing and transport device 20, in which access to the internal cavity 20′ is prevented, to an open position to allow accessing the internal cavity 20′, and vice versa.

The aforesaid reversible coupling means of the housing and transport device 20 comprise a pin 26 protruding, in an off-center position, in the aforesaid internal housing cavity 20′. Such a pin 26 also operates as an element for fixing the orientation 26′ of the endoscope 1.

Moreover, the housing and transport device 20 comprises inside the housing body 24 of the endoscope 1, an element 81 for displaying one or more images representative of calibration values associated with the one or more sensors of the endoscope 1. In particular, the images reproduced on said display element 81 are unique graphic representation identification codes which include the calibration values of the sensors 2, 3, 4, 6 of the endoscope 1 in a machine-readable format. In particular, such unique graphic identification codes can be one-dimensional codes such as barcodes, or two-dimensional codes of the Data Matrix or QR-code type.

With reference to the figures, the housing and transport device in the closing configuration comprises a through hole 50 to allow the passage of the electric cables F connecting the endoscope 1 to the robotic platform 60 of the system 200. In general, note that such a through hole allows the passage of electrical, hydraulic and pneumatic connections between the base and the tip of the endoscope.

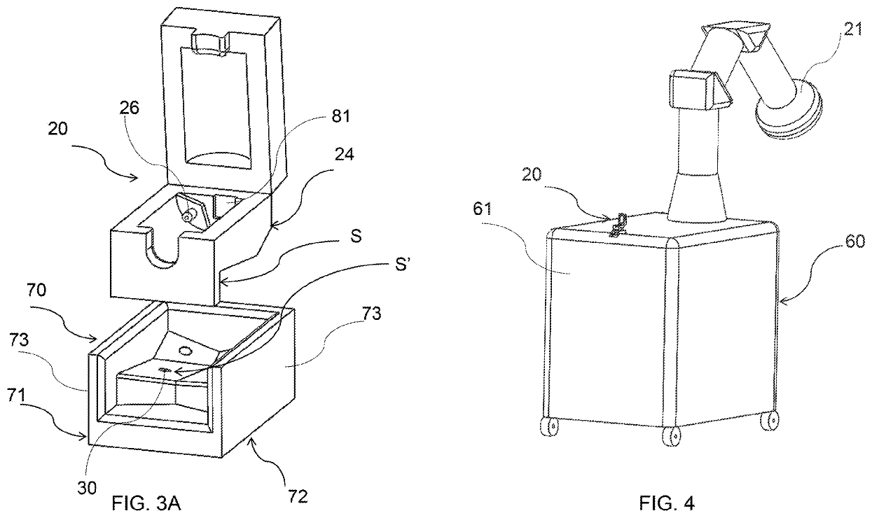

With reference to the embodiment in FIGS. 3a-3c, the endoscopic robotic system 200 comprises an interface element 70 housed in the robotic platform 60.

In an embodiment, the body 24 of the housing and transport device 20 comprises the above-mentioned coupling surface S configured to engage a first coupling surface S′ obtained in such an interface element 70 to achieve a mechanical shape coupling with such an interface element 70.

In an alternative embodiment, the endoscopic robotic system 200 comprises an interface element 70 configured to receive the body 24 of the housing and transport device 20. A first coupling surface S′ of such an interface element 70 comprises at least one magnetic element 30 configured to achieve a magnetic coupling between the interface element 70 and the housing and transport device 20, locking said housing and transport device 20 accommodated in such an interface element 70.

In a further embodiment, the body 24 of the housing and transport device 20 comprises the coupling surface S configured to engage the first coupling surface S′ of the interface element 70, comprising the at least one magnetic element 30, to also make a mechanical shape coupling with such a first surface S′.

Note that the aforesaid magnetic element 30 is a permanent magnet configured to operate as a magnetic field source 23 housed in the base portion 61 of the robotic platform 60.

In the example in FIGS. 2A, 2B, 2C, the coupling surface S of the housing and transport device 20 which is opposite to the second portion 24b of the body 24b, is a continuous surface delimited by a first wall S1 orthogonal to a longitudinal axis of the first body portion 24a. Such a coupling surface S further comprises a second wall S2 inclined along such a longitudinal axis from the first wall S1, adapted to define with such a longitudinal axis a first inclination in a first direction. Such a coupling surface S also comprises a third inclined wall S3 adapted to define with such a longitudinal axis a second inclination in a second direction opposite to the first direction.

With reference to FIGS. 3A, 3B, 3C, 5, 6, such an interface element 70 has a respective body 71 in the shape of a prism, for example with a rectangular or square base, comprising a base wall 72 connected to two major side walls 73 and to a minor side wall 74. Such major 73 and minor 74 side walls are orthogonal to the base portion 72 to delimit a first cavity 75 for inserting the housing and transport device 20 into the interface element 70. In particular, the first cavity 75 is delimited by the aforesaid first surface S′ shaped as a hollowed step comprising walls configured to adapt to the first S1, second S2 and third S3 walls of the surface of the device 20 described above so as to achieve a mechanical shape coupling with such a surface S.

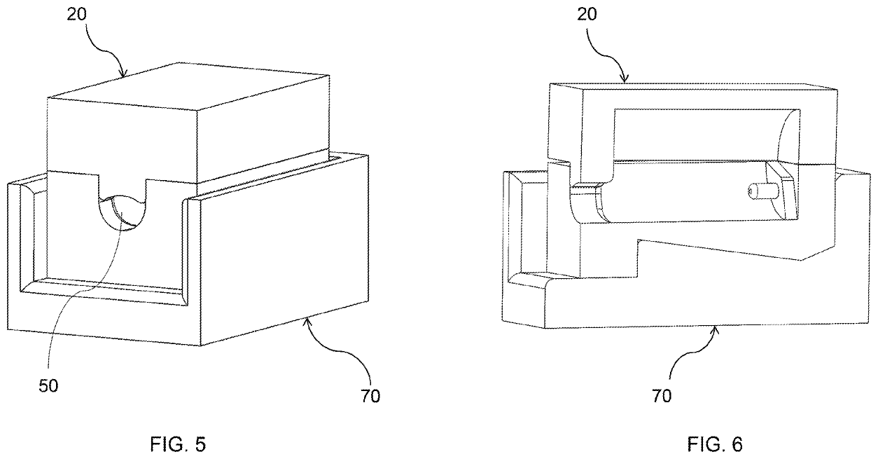

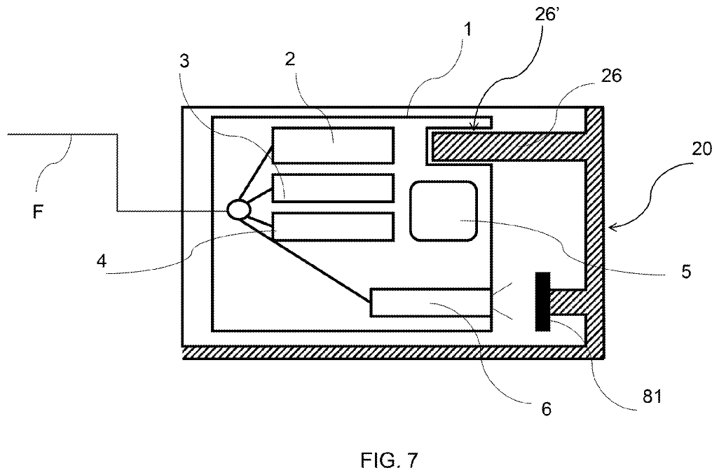

FIGS. 5 and 6 show, in front perspective view and in section, an assembly of the interface element 70 housable in the robotic platform 60 and of the housing and transport device 20 of the endoscope 1, in which such a housing and transport device 20 is coupled to the interface element 70.

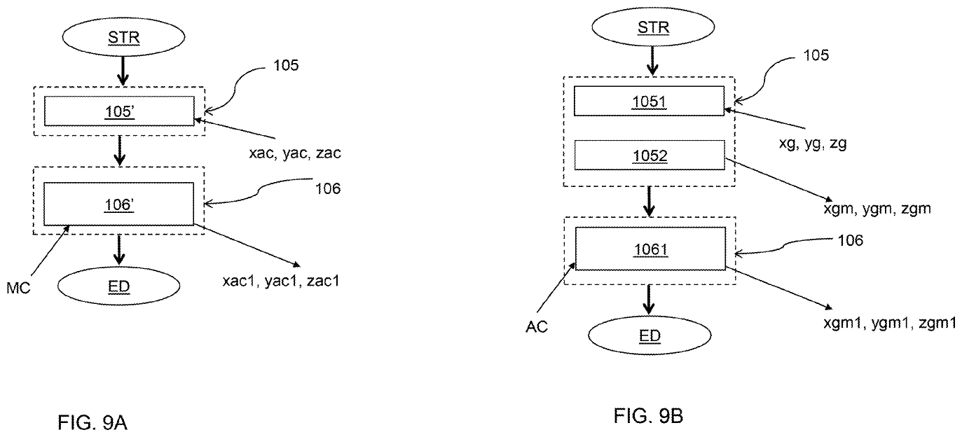

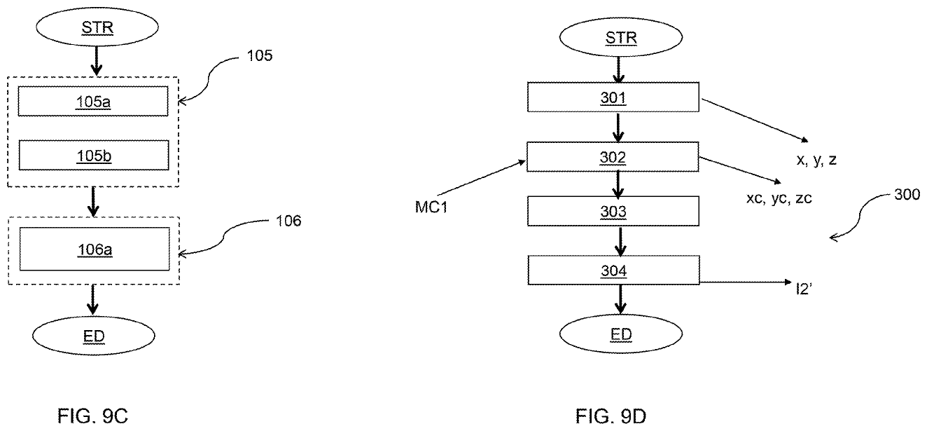

With reference to FIG. 8, a general embodiment of a method 100 for transporting and verifying calibration values associated with sensors accommodated in an endoscope 1 is shown with a flow diagram.

The method starts with a symbolic start step STR and ends with a symbolic end step ED and in the most general form can be implemented with the system 200 described above before use of the endoscope 1.

In an embodiment, the electronic processing unit 25 of the system 200 is arranged to execute the codes for an application program implementing the method 100 of the present invention.

In particular, the microprocessor of such a processing unit is configured to load, in a respective memory block, and execute the codes of the application program implementing the method 100 of the present invention.

The method for transporting and verifying calibration values or simply method 100 comprises an initial step of providing 101 an endoscopic robotic system 200 comprising:
an endoscope 1 including one or more sensors 2, 3, 4, 6;
a magnetic field source 21, 23 for providing a reference magnetic field value to the endoscope 1;
an electronic processing unit 25;
a reading unit 22 connected to the endoscope 1 for acquiring data representative of measurements made by such one or more sensors of the endoscope to be sent to the electronic processing unit 25;
a reading unit 22′ of calibration values associated with the aforesaid one or more sensors of the endoscope 1, connected to the endoscope 1 to acquire the calibration values to be sent to the electronic processing unit 25.

The method 100 further comprises the step of providing 102 a device for housing and transporting 20 the endoscope having a body 24 adapted to house the endoscope 1 and a step of coupling and making integral 103 the endoscope 1 with the body 24 of the housing and transport device 20 preventing a mutual movement thereof.

The method 100 includes placing 104 the housing and transport device 20 in a predetermined position of the endoscopic robotic system 200 to provide the endoscope 1 with the aforesaid reference magnetic field value generated by the magnetic field source 21, 23.

The method includes a step of acquiring 105, by the electronic processing unit 25:
the calibration values associated with the one or more sensors 2, 3, 4, 6 of the endoscope 1,
first data representative of measurements made by the one or more sensors 2, 3, 4, 6 when the reference magnetic field value is applied to the endoscope 1 in the predetermined position of the endoscopic robotic system 200;
reference data representative of correct measurements made by the one or more sensors 2, 3, 4, 6 when the reference magnetic field value is applied to the endoscope 1 in the predetermined position of the endoscopic robotic system 200.

The method includes a step of applying 106 the calibration values to the first acquired data to generate first calibrated data.

It is then possible to compare 107 the first calibrated data with the reference data.

Based on such a comparison, the method 100 includes returning 108, by the electronic processing unit 25:
first information I1, indicating that the calibration values associated with the one or more sensors 2, 3, 4, 6 of the endoscope 1 are correct,
or
second information I2 indicating that the calibration values associated with the one or more sensors 2, 3, 4, 6 of the endoscope 1 are incorrect.

In particular, such first information I1 indicates that one or more of the first calibrated data coincides with one of the reference data at less than a predetermined tolerance value. Moreover, the second information I2 indicates that one or more of the first calibrated data differs from one of the reference data by values greater than a predetermined tolerance value.

In a particular embodiment, the aforesaid first I1 and second I2 information are binary information.

Particular embodiments of the method 100 of the invention are described below, if the calibration values to be verified are those of the accelerometer 2, the gyroscope 3 and the magnetic field sensors 4. The method steps related to such particular examples can be performed independently for each type of sensor or concomitantly.

In the embodiment where such one or more sensors of the endoscope 1 comprise an accelerometer 2,
the acquiring step 105 of the method 100 described above comprises a step of recording 105′, at a predetermined time instant, the first accelerometer data 2 consisting of a triad of accelerometer values xac, yac, zac, each value of the triad being measured with reference to an axis of an orthogonal Cartesian reference system of the accelerometer 2;
the applying step 106 of the method 100 comprises a step of multiplying 106′ the triad of first accelerometer data xac, yac, zac by a calibration values matrix MC, in particular three rows and three columns, to generate a triad of first calibrated accelerometer data xac1, yac1, zac1.

Based on a comparison of such first calibrated accelerometer data xac1, yac1, zac1 with the reference data, the method 100 includes a step of providing, by the electronic processing unit 25, the second information I2 indicating that the calibration values associated with the accelerometer 2 are incorrect when one or more of said first calibrated accelerometer data xac1, yac1, zac1 differs from one of the reference data by values greater than a predetermined tolerance value.

In the embodiment in which the one or more sensors of the endoscope 1 comprise a gyroscope 3, the acquiring step 105 of the method comprises the further steps of:
recording 1051, at a predetermined time instant, the first gyroscope data 3 consisting of a triad of gyroscope values xg, yg, zg, where each value of the triad is measured with reference to an axis of an orthogonal Cartesian reference system of the gyroscope 3,
calculating 1052 an average of all the values recorded for each axis to generate a triad of first average gyroscope 3 data xgm, ygm, zgm.

Moreover, the applying step 106 of the method 100 comprises a step of subtracting 1061 from the triad of first average gyroscope data xgm, ygm, zgm an array of calibration values AC, to generate a triad of first calibrated gyroscope data xgm1, ygm1, zgm1. Based on a comparison of such first calibrated gyroscope data xgm1, ygm1, zgm1 with said reference data, there is included a of step providing, by the electronic processing unit 25, the second information I2 indicating that the calibration values associated with the gyroscope 3 are incorrect when one or more of such first calibrated gyroscope data xac1, yac1, zac1 differs from zero by values greater than a predetermined tolerance value.

In the embodiment in which the one or more sensors of the endoscope 1 comprise one or more magnetic field sensors 4, the aforesaid acquiring step 105 of the method 100 comprises the steps of:
recording 105a, for each magnetic field sensor 4, a plurality of first magnetic field sensor data 4,
calculating 105b, for each sensor 4, an average of all the first recorded data to generate a first magnetic field sensor datum 4 with average value.

The aforesaid applying step 106 of the method 100 comprises, for each sensor 4, a step of subtracting 106a from the first magnetic field sensor datum 4 with average value a calibration number associated with such a sensor, to generate a calibrated first magnetic field sensor datum 4 with average value.

Based on a comparison, for each sensor 4, of said such a calibrated first magnetic field sensor datum having an average value with the reference datum, there is included a step of providing, by the electronic processing unit 25, the second information I2 indicating that the calibration value associated with the magnetic field sensor 4 is incorrect when the difference between the calibrated first magnetic field sensor datum 4 with average value and such a reference datum is different from a predetermined number.

In an optional embodiment, again with reference to the one or more magnetic field sensors 4 of the endoscope, the method 100 of the invention comprises a further step of verifying 300 an alignment of such magnetic field 4. In particular, sensors such an alignment verification step 300 comprises the steps of:
grouping 301 the calibrated first magnetic field sensor datum 4 with average value acquired for each sensor 4 to generate one or more triads x, y, z of calibrated first average value data each of which is associated with one of the axes of the magnetic field sensors 4;
multiplying 302 each of such triads of calibrated first average value data by a first calibration value matrix MC1, in particular three rows and three columns, to generate a further calibrated triad xc, yc, zc;
comparing 303 the values of said further calibrated triad xc, yc, zc, representative of the direction of the reference magnetic field in the position of the sensor 4, with reference data;
providing, by the electronic processing unit 25, based on such a comparison, for each sensor 4, further second information I2′ indicating that the calibration value associated with the magnetic field sensor 4 is incorrect when the difference between at least one of the values of such a further calibrated triad xc, yc, zc and such reference data is different from a predetermined number.

The present invention also relates to a housing and transport device 20 of an endoscope 1 included in a magnetically guided endoscopic robotic system 200. Such a housing and transport device comprises:
a body 24 adapted to house the endoscope 1;
reversible coupling means 26, 26′ for reversibly coupling the endoscope 1 to the body 24 of the housing and transport device 20, configured to couple and make integral the endoscope 1 with the body of the housing and transport device 20 preventing a mutual movement thereof.

Moreover, the body 24 of the housing and transport device 20 comprises a coupling surface S configured to engage, by a mechanical shape coupling, the magnetically guided endoscopic robotic system 200.

The method 100 for transporting and verifying the calibration values associated with sensors accommodated in an endoscope 1 and the housing and transport device 20 of an endoscope of the present invention have several advantages and achieve the intended objects.

In particular, the method 100 ensures, in a simple and reliable manner, the transport of the calibration values of the sensors 2, 3, 4 with the endoscope 1, for example included in a respective memory, for example solid state memory, or on another support associated with the body 24 of the housing and transport device 20.

Moreover, the method 100 ensures the communication of the same calibration values to the processing unit 25 of the endoscopic robotic system 200, for example through the camera 6 of the endoscope. In fact, such a device 20 comprises an element 81 for displaying one or more images representative of calibration values associated with the one or more sensors of the endoscope 1. Such images of the element can be read through the camera 6 of the endoscope so that the system 200 can access the calibration values automatically.

In addition, the method 100 ensures verification of the validity of such calibration values before using the endoscope 1. In particular, if the verification is successful, the endoscope 1 is usable, otherwise it needs recalibration or must be disposed of, as it is not usable.

Moreover, the housing and transport device 20 ensures the cleaning of the endoscope 1 during the pre-operating steps and protects the endoscope 1 during transport.

Moreover, with reference to the hinges mentioned above, the device of the invention is configured to indicate whether the endoscope is usable as it has not been used before.

In order to meet contingent needs, those skilled in the art may make changes and adaptations to the embodiments of the method described above or can replace elements with others which are functionally equivalent, without departing from the scope of the following claims. Each of the features described above as belonging to one possible embodiment can be implemented irrespective of the other embodiments described.

1-21. (canceled)

22. A method for transporting and verifying calibration values associated with sensors accommodated in an endoscope usable in a magnetically guided endoscopic system, comprising the steps of:
providing an endoscopic robotic system comprising:
an endoscope including one or more sensors;
a magnetic field source for providing a reference magnetic field value to the endoscope;
an electronic processing unit;
a reading unit connected to the endoscope to acquire data representative of measurements made by said one or more sensors of the endoscope to be sent to the electronic processing unit;
a reading unit of calibration values associated with said one or more sensors of the endoscope, connected to the endoscope to acquire said calibration values to be sent to the electronic processing unit;
said method further comprising the steps of:
providing an endoscope housing and transport device having a body adapted to house said endoscope;
coupling and making integral the endoscope with the body of the housing and transport device, preventing a mutual movement thereof;
placing said housing and transport device in a predetermined position of the endoscopic robotic system to provide the endoscope with said reference magnetic field value generated by the magnetic field source;
acquiring, by said electronic processing unit:
the calibration values associated with said one or more sensors of the endoscope;
first data representative of measurements made by said one or more sensors when the reference magnetic field value is applied to the endoscope in said predetermined position of the endoscopic robotic system;
reference data representative of correct measurements made by said one or more sensors when the reference magnetic field value is applied to the endoscope in said predetermined position of the endoscopic robotic system;
applying said calibration values to the first acquired data to generate first calibrated data;
comparing said first calibrated data with said reference data;
returning, by said electronic processing unit, based on said comparison:
first information, indicating that the calibration values associated with said one or more sensors of the endoscope are correct,
or
second information indicating that the calibration values associated with said one or more sensors of the endoscope are incorrect.

23. The method for transporting and verifying calibration values associated with sensors accommodated in an endoscope according to claim 22, wherein:
the first information indicates that one or more of said first calibrated data coincides with one of said reference data at less than a predetermined tolerance value,
the second information indicates that one or more of said first calibrated data differs from one of said reference data for values greater than a predetermined tolerance value.

24. The method for transporting and verifying calibration values associated with sensors accommodated in an endoscope according to claim 22, wherein said first and second information are binary-type information.

25. The method for transporting and verifying calibration values associated with sensors accommodated in an endoscope according to claim 22, wherein said step of coupling and making integral the endoscope with the body of the housing and transport device comprises a step of providing coupling means for reversibly coupling the endoscope to the body of the housing and transport device.

26. The method for transporting and verifying calibration values associated with sensors accommodated in an endoscope according to claim 22, wherein said magnetic field source is a permanent magnet housed in a base portion of a robotic platform of the endoscopic robotic system.

27. The method for transporting and verifying calibration values associated with sensors accommodated in an endoscope according to claim 22, wherein said magnetic field source is a magnetic field source controllable by the electronic processing unit fixed to one end of a robotic arm of a robotic platform of the endoscopic robotic system.

28. The method for transporting and verifying calibration values associated with sensors accommodated in an endoscope according to claim 22, wherein said step of placing the housing and transport device in a predetermined position of the endoscopic robotic system comprises a step of providing means for removably coupling the housing and transport device to a robotic platform of the endoscopic robotic system in said predetermined position.

29. The method for transporting and verifying calibration values associated with sensors accommodated in an endoscope according to claim 28, wherein said removable coupling means are mechanical, magnetic, or a combination of both.

30. The method for transporting and verifying calibration values associated with sensors accommodated in an endoscope according to claim 28, further comprising a step of providing an interface element housed in the robotic platform of the endoscopic robotic system, the body of the housing and transport device comprising a coupling surface configured to engage a first coupling surface obtained in said interface element to achieve a mechanical shape coupling with said interface element.

31. The method for transporting and verifying calibration values associated with sensors accommodated in an endoscope according to claim 28, further comprising a step of providing an interface element housed in the robotic platform of the endoscopic robotic system configured to accommodate the body of the housing and transport device, a first coupling surface of said interface element comprising at least one magnetic element configured to achieve a magnetic coupling between the interface element and the housing and transport device, locking said housing and transport device accommodated in said interface element.

32. The method for transporting and verifying calibration values associated with sensors accommodated in an endoscope according to claim 31, wherein the body of the housing and transport device comprises a coupling surface configured to engage the first coupling surface of said interface element to achieve a mechanical shape coupling.

33. The method for transporting and verifying calibration values associated with sensors accommodated in an endoscope according to claim 22, wherein said one or more sensors of the endoscope comprise:
an accelerometer,
a gyroscope,
one or more magnetic field sensors.

34. The method for transporting and verifying calibration values associated with sensors accommodated in an endoscope according to claim 33, wherein said one or more sensors of the endoscope further comprise a camera.

35. The method for transporting and verifying calibration values associated with sensors accommodated in an endoscope according to claim 33, wherein, when said one or more sensors of the endoscope comprise an accelerometer,
said acquiring step comprises a step of recording, at a predetermined time instant, said first accelerometer data consisting of a triad of accelerometer values, each triad value being measured with reference to an axis of an orthogonal Cartesian reference system of the accelerometer;
said applying step comprises a step of multiplying said triad of first accelerometer data by a calibration values matrix with three rows and three columns, to generate a triad of first calibrated accelerometer data;
based on a comparison of said first calibrated accelerometer data with said reference data, there is included a step of providing, by said electronic processing unit, the second information indicating that the calibration values associated with said accelerometer are incorrect when one or more of said first calibrated accelerometer data differs from one of the reference data for values greater than a predetermined tolerance value.

36. The method for transporting and verifying calibration values associated with sensors accommodated in an endoscope according to claim 33, wherein, when said one or more sensors of the endoscope comprise a gyroscope,
said acquiring step comprises the steps of:
recording, at a predetermined time instant, said first gyroscope data consisting of a triad of gyroscope values, each triad value being measured with reference to an axis of an orthogonal Cartesian reference system of the gyroscope,
calculating an average of all the values recorded for each axis to generate a triad of first average gyroscope data;
said applying step comprises a step of subtracting from said triad of first average gyroscope data an array of calibration values, to generate a triad of first calibrated gyroscope data;
based on a comparison of said first calibrated gyroscope data with said reference data, there is included a step of providing, by said electronic processing unit, the second information indicating that the calibration values associated with said gyroscope are incorrect when one or more of said first calibrated gyroscope data differs from zero for values greater than a predetermined tolerance value.

37. The method for transporting and verifying calibration values associated with sensors accommodated in an endoscope according to claim 33, wherein, when said one or more sensors of the endoscope comprise one or more magnetic field sensors,
said acquiring step comprises the steps of:
recording, for each magnetic field sensor, a plurality of first magnetic field sensor data,
calculating, for each sensor, an average of all the first recorded data to generate a first magnetic field sensor datum with average value;
said applying step comprises, for each sensor, a step of subtracting from said first magnetic field sensor datum with average value a calibration number associated with said sensor, to generate a calibrated first magnetic field sensor datum with average value;
based on a comparison, for each sensor, of said calibrated first magnetic field sensor datum having an average value with said reference datum, there is included a step of providing, by said electronic processing unit, the second information indicating that the calibration value associated with said magnetic field sensor is incorrect when the difference between the calibrated first magnetic field sensor datum with average value and said reference datum is different from a predetermined number.

38. The method for transporting and verifying calibration values associated with sensors accommodated in an endoscope according to claim 37, further comprising a further step of verifying an alignment of the magnetic field sensors of the endoscope, said step comprising the steps of:
grouping said calibrated first magnetic field sensor data with average value acquired for each sensor to generate one or more triads of calibrated first average value data each of which is associated with one of the axes of the magnetic field sensors;
multiplying each of said triads of calibrated first average value data by a first calibration value matrix with three rows and three columns, to generate a further calibrated triad;
comparing the values of said further calibrated triad, representative of the direction of the reference magnetic field in the position of the sensor, with reference data;
providing, by said electronic processing unit, based on said comparison, for each sensor, further second information indicating that the calibration value associated with said magnetic field sensor is incorrect when the difference between at least one of the values of said further calibrated triad and said reference data is different from a predetermined number.

39. A magnetically guided endoscopic robotic system, comprising:
an endoscope including one or more sensors;
a magnetic field source to provide a reference magnetic field value to the endoscope;
an electronic processing unit;
a reading unit connected to the endoscope to acquire data representative of measurements made by said one or more sensors of the endoscope to be sent to the electronic processing unit;
a reading unit of calibration values associated with said one or more sensors of the endoscope, connected to the endoscope to acquire said calibration values to be sent to the electronic processing unit;
a device for housing and transporting the endoscope having a body adapted to house said endoscope, the endoscope being coupled and made integral with the body of the housing and transport device to prevent a mutual movement thereof;
means for placing the housing and transport device in a predetermined position of the endoscopic robotic system to provide the endoscope with said reference magnetic field value generated by the magnetic field source,
said system being configured to perform the method for transporting and verifying calibration values associated with the sensors accommodated in the endoscope according to claim 22.

40. A housing and transport device of an endoscope, said housing and transport device comprising:
a body adapted to house said endoscope;
reversible coupling means for reversibly coupling the endoscope to the body of the housing and transport device, configured to couple and make integral the endoscope with the body of the housing and transport device preventing a mutual movement thereof;
said body of the housing and transport device comprising a coupling surface configured to engage, by a mechanical shape coupling, a magnetically guided endoscopic robotic system according to claim 39,
wherein the body of the housing and transport device has a box-like shape to delimit an internal cavity for housing the endoscope, said body comprising a first and a second body portion, the first body portion having a first end connected by a hinge to a respective first end of the second body portion, each of said first and second body portions having a respective second free end opposite to said first end,
said hinge allowing a mutual movement of the first and second body portions between a closing position of the housing and transport device to an opening position to allow accessing said internal cavity, and vice versa.

41. The housing and transport device of an endoscope according to claim 40, wherein said reversible coupling means comprise a pin protruding into the aforesaid internal housing cavity.

42. The housing and transport device of an endoscope according to claim 40, further comprising, inside the housing body of the endoscope, an element for displaying one or more images representative of calibration values associated with said one or more sensors of the endoscope.
